(19) United States
(12) Patent Application Publication
(10) Pub. No.: US 2006/0085037 A1
(43) Pub. Date: Apr. 20, 2006

(54) Electro-alysis

(76) Inventor: Robert J. Lemire, Kings Park, NY (US)

Correspondence Address: ROBERT J. LEMIRE, P.O. BOX 299, KINGS PARK, NY, 11754, US

(21) Appl. No.: 10/964,018

(22) Filed: Oct. 14, 2004

(51) Int. Cl. A61N 1/00

(52) U.S. Cl. 607/2

(57) ABSTRACT

An electrical shock process is applied to a person for the deactivation of biological contaminates within the tissue and blood of the person. A shock array of electrodes is placed on the person and a series of shocks is applied to the desired site. The shocks penetrate the tissue and blood where they interact with and deactivate various contaminates. Small shock arrays applied to the skin can be used for the treatment of insect bites while larger ones are used for the topical treatment of the entire being. Internally implanted shock arrays can also be used for the direct treatment of blood and vital organs.

### CROSS-REFERENCE TO RELATED APPLICATIONS

(Not Applicable)

### STATEMENT RE: FEDERALLY SPONSORED RESEARCH/DEVELOPMENT

(Not Applicable)

### BACKGROUND OF THE INVENTION

The present invention generally relates to improvements to biological electrical shock therapy that renders internal biological contaminants incapable of acting on humans and animals.

Prior inventions have detailed the effects that electrical shocks have on the body; however, none have been specifically applied to the deactivation of internal biological contaminants that can negatively affect the organism.

On Aug. 5, 1986 the NY Times published an article titled “New Shock Therapy for Snakebites”. This article detailed how the application of high voltage shocks could eliminate the effects of venomous snakebites in humans. Subsequent to this pronouncement several respected laboratories performed experiments to substantiate the claims. Web site www. Kamakazi.com/docsplace/aoi/snakebite.html has an article titled “Debunking Usage of Electric Shock for First-Aid Treatment of Venomous Snakebite—How the Electric Snakebite Treatment Craze Got Started”. This article has several references to the testing that was performed. Therefore, research on, and the use of electrical shocks for the treating of snakebites has been abandoned.

In spite of the lab results this applicant has successfully been using electrical shocks to eliminate the effects of mosquito bites and bee stings. The applicant believes that the labs performing the testing for snakebites did not follow a procedure that would lead to a satisfactory result. That is, they may have used a single shock of an inappropriate voltage, or any other combination of variables.

This application is for the process that uses electrical shocks to deactivate biological contaminants within a person and any number of devices utilizing that process for the purpose of eliminating internal biological contaminants.

### BRIEF SUMMARY OF THE INVENTION

When an insect bites a person or animal it injects biological components that produce a harmful effect on the person that was bit. The application of repeated electrical shocks at and around the site of the bite have been found to rapidly eliminate the effects of the bite. That is; the pain, the itch, and the swelling that accompany such bites are eliminated in a very short period of time after the application of the electrical shocks. It is therefore concluded that the electrical shocks were able to deactivate the contaminating biological material. This deactivation is not limited to material injected by insects. Electrical shocks will deactivate a wide range of biological contaminants within the body. This fact then leads to a number of devices that, when activated, provide a number of shocks in quick succession to a target area on a person. Contaminating biological components present in the surrounding area of the person are then deactivated and rendered ineffectual by this action.

There are several products that currently apply electrical shocks to humans. These can be for muscle stimulation or for regulating a heartbeat. These devices are actually benefiting the users in this new and heretofore unrecognized way. The shocks delivered by these devices are actually eliminating biological contaminants as a side effect of their use; however, the level and frequency of the shocks is too small to produce a noticeable effect on the person.

The process and any number of devices for delivering electrical shocks for the purpose of deactivating biological contaminants is what is being claimed in this application.

Such devices include, but are not limited by the following:
1. Small battery-powered units for delivering shocks to the site of insect bites
2. Large units for delivering electrical shocks to one or more large areas of the body such as the legs, the back, and the arms
3. Internally implanted units applied to the surface of major veins and arteries, or to organs such as the liver and kidneys.

### BRIEF DESCRIPTION OF THE DRAWINGS

FIG. 1 is a schematic of the major components used to implement the process.

### DETAILED DESCRIPTION OF THE INVENTION

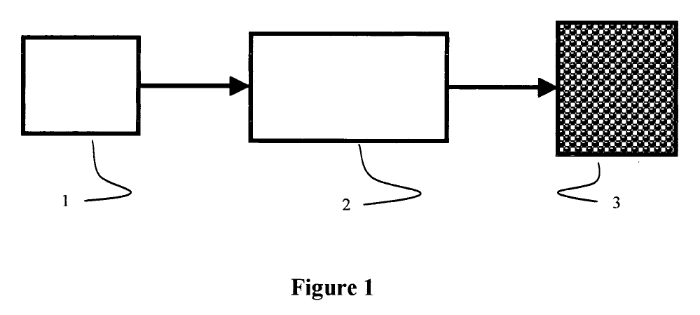

Referring now to the drawing wherein the showings are for purposes of illustrating a preferred embodiment of the present process only, and not for purposes of limiting the same, FIG. 1 illustrates power supply 1 feeding shock distribution system 2 that regulates the sequencing of the shocks sent to the shock array 3 that is placed on the person. Activation of the distribution system 2 causes a predetermined series of shocks to be sent to the shock array 3. The shock array 3 is applied to a desired area of the body of the person being treated. The sequencing shocks from the shock array 3 penetrate the tissue and deactivate the harmful capabilities of biological contaminants that may be present in the tissue receiving the shocks.

In the case of insect bites a battery is used for the power supply 1, and the shock array 3 is positioned on the skin over the bite. The application of the shocks by activating the distribution system 2 deactivates the biological contaminants injected into the body by the insect.

When a major contamination of a body is to be treated externally, the power supply 1 is a regulated source and the shock array 3 covers a large area of the body. Again the distribution system applies a series of shocks to the person for a specific time. The intensity of the shocks and the duration is set to accomplish a desired degree of treatment. Likewise, shock arrays 3 can be implanted so as to treat internal sites, such as major veins and arteries, and organs like the liver and kidneys.

Additional modifications and improvements of the embodiments of the present invention may also be apparent to those of ordinary skill in the art. Thus, the particular combination of parts described and illustrated herein is intended to represent only a certain embodiment of the present process, and is not intended to serve as limitations of alternative devices within the spirit and scope of the invention.

What is claimed is:

1. A process for deactivating biological contaminants within the body of a being by the application of a series of electrical shocks over a period of time.

2. The process of claim 1 wherein a series of electrical shocks are delivered to an area on the surface of the skin, said shocks penetrating the underlying tissue and blood, and in the process deactivate contaminants present in the underlying tissue and blood, thereby allowing the body to recover from and avoid further damage from the contaminants.

3. The process of claim 2 wherein an electrical power source produces a voltage that is transmitted to a distribution system that selects alternating pairs of electrodes on a shock array placed on the surface of the body, wherein the resulting shocks throughout the shock array deactivate contaminants in the tissue and blood beneath the shock array.

4. The process of claim 3 wherein the shock array is placed on internal organs, whereby biological contaminants within these organs are deactivated by the shocks.

5. The process of claim 3 wherein the shock array is placed on internal arteries and veins for deactivating contaminants within the blood stream.
